(19) United States
(12) Patent Application Publication
(10) Pub. No.: US 2005/0124839 A1
(43) Pub. Date: Jun. 9, 2005

(54) Catalyst and process for the metathesis of ethylene and butene to produce propylene

(76) Inventors: Robert J. Gartside, Summit, NJ (US); Marvin I. Greene, Hackensack, NJ (US)

Correspondence Address: DILWORTH & BARRESE, LLP, 333 EARLE OVINGTON BLVD., UNIONDALE, NY, 11553, US

(21) Appl. No.: 10/983,449

(22) Filed: Nov. 8, 2004

Related U.S. Application Data
(63) Continuation-in-part of application No. 10/705,270, filed on Nov. 10, 2003, now abandoned.
Application No. 10/705,270 is a division of application No. 09/880,670, filed on Jun. 13, 2001, now Pat. No. 6,683,019.

(51) Int. Cl. C07C 6/00

(52) U.S. Cl. 585/643

(57) ABSTRACT

A process for producing propylene from a C₄ feed containing 2-butene includes contacting said feed with ethylene in a metathesis reaction zone containing a metathesis catalyst under metathesis reaction conditions to provide an effluent including propylene, said metathesis catalyst consisting essentially of a transition metal or oxide thereof supported on a high purity silica support possessing less than about 150 ppm magnesium, less than about 900 ppm calcium, less than about 900 ppm sodium, less than about 200 ppm aluminum, and less than about 40 ppm iron.

### CROSS-REFERENCE TO RELATED APPLICATIONS

The present application is a continuation-in-part of copending U.S. application Ser. No. 10/705,270 filed Nov. 10, 2003, which is a divisional of U.S. application Ser. No. 09/880,670 filed Jun. 13, 2001, to which priority is claimed and which is now issued as U.S. Pat. No. 6,683,019 B2.

### BACKGROUND

1. Field of the Invention

The present invention relates to a supported catalyst for the metathesis, or disproportionation, of olefin(s), and to metathesis processes employing the catalyst.

2. Background of the Art

The metathesis, or disproportionation, of olefin(s) is a reaction in which one or more olefinic compounds are transformed into other olefins of different molecular weights. The disproportionation of an olefin with itself to produce an olefin of a higher molecular weight and an olefin of a lower molecular weight is also referred to as self-disproportionation. For example, propylene can be disproportionated to ethylene and cis- and trans-2-butene. Another type of disproportionation involves the cross-disproportionation of two different olefins to form still other olefins. An example would be the reaction of one molecule of 2-butene with one molecule of 3-hexene to produce two molecules of 2-pentene.

When olefins are contacted with metathesis catalysts, the reactions proceed according to a specific structural relationship depending upon the character of the feedstock. The reaction is generally considered to proceed using a four-centered active site on the catalyst. The olefinic double bonds line up on opposite sides of the four-centered site. The reaction proceeds under equilibrium conditions with the bonds exchanging sides of the four-centered site and thusly exchanging the hydrocarbon groups attached to one end of the double bond with the groups attached to the other olefin. For example, 2-butene if reacted with ethylene can form two propylene molecules as shown by equation (1) where each corner of each box in equation (1) represents one of the four active sites on the catalyst:

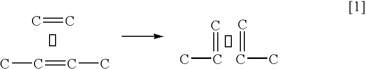

Extending this concept to any number of olefins, one can see that depending upon the nature of the R group attached to the double bonds, different olefins are formed with strict adherence to the exchange of R groups around the double bond. Thus, olefin R1—C═C—R2 when reacted with olefin R3—C═C—R4 forms an olefin of R1—C═C—R3 and an olefin of R2—C═C—R4.

One skilled in the art can imagine many potential reactions over the entire range of possible olefin pairs. Importantly however, an alpha olefin and ethylene will not react with each other under metathesis conditions. There is no reaction between 1-butene and ethylene for example but considerable reaction between 2-butene and ethylene.

In addition to the metathesis reactions, however, it is not uncommon for various side reactions to occur. One such reaction is an oligomerization reaction where olefins combine to form larger olefins. This reaction, if the olefin grows large enough, leads to fouling of the catalyst as the active sites are blocked. Another reaction that could occur is the double bond isomerization of the olefin. In this case, the position of the double bond shifts within the hydrocarbon chain. Examples are the isomerization of 1-butene to 2-butene and 3-hexene to 2-hexene. If this occurs, the number and character of the olefins available for metathesis changes. With olefins having different R groups available, different reaction products can be formed. The isomerization side reaction leads to a loss in the selectivity of the metathesis reaction to the products defined by the structure of the feedstock olefins.

In some process configurations, a mixture of 1 butene and 2 butene is fed to a metathesis unit along with ethylene to produce propylene. In this case, the 2 butene will react with ethylene to form propylene but the 1 butene will not react with ethylene. It is not unusual in these conditions to include a isomerization function to isomerize 1-butene to 2-butene in order to allow for the maximum conversion of the normal butenes (both 1- and 2-butene). This activity can be either in the form of a commixed isomerization catalyst (such as MgO) or alternately as highly acidic or basic sites on the metathesis catalyst.

For example, if the feedstock to the metathesis reaction was essentially pure 1-butene, the primary products of that reaction would be ethylene and 3-hexene. No other products would form. If, however, some portion of the 1-butene was isomerized to 2-butene, then 1-butene could react with 2-butene to form propylene and 2-pentene. The formation of 2-pentene in stepwise fashion allows for the formation of higher carbon number olefins, e.g., 2-pentene can react with 1-butene to form 3-hexene and propylene. The propylene, 2-pentene and 3-hexene represent non-selective products. Similarly if a feed of essentially pure 2 butene was to be reacted with ethylene to form 2 propylenes, should any isomerization of the 2 butene to 1 butene occur, then the reaction between 1 butene and 2 butene would yield propylene and 2 pentene creating selectivity losses.

The ability to control unwanted side reactions allows the process designer to selectively produce specific products based upon the purity and character of the feedstocks. In many cases this is important to maximize the value of a particular reaction. An example of such a process where selectivity is critical is the production of linear alpha olefins as described in commonly assigned, U.S. Pat. No. 6,727,396, incorporated by reference herein. That process requires a catalyst with low isomerization activity as described therein.

Many catalysts have been developed for metathesis. For example, those comprising inorganic oxides containing a catalytic amount of a metal or metal oxide have been employed widely for continuous, fixed-bed conversion of olefins. One such catalyst comprises a silica support and an oxide of tungsten. The present invention is based on the discovery of a way to improve the selectivity of metathesis catalysts to specific products, particularly propylene, which is of high commercial value.

Propylene is produced by the metathesis of ethylene and 2-butene. In this system typically a high ethylene to butenes ratio is used in order to minimize the reactions between 1- and 2-butene. The 1-butene can be formed from isomerization activity of the metathesis catalyst. The reaction between 1-butene and 2-butene forms propylene and 2-pentene. It is preferable to have a low ethylene to butene feed ratio to the metathesis reactor to reduce the expensive recycling of ethylene separated from the metathesis effluent. However lower ethylene ratios when utilizing non-selective metathesis catalysts result in some isomerization of 2-butene to 1-butene and the subsequent reduction in the selectivity to propylene from the formation of pentenes and hexenes, which are of lesser commercial value than propylene. Accordingly, a more efficient process for the manufacture of propylene by ethylene-butene metathesis is needed.

### SUMMARY

A process is provided for producing propylene from a C₄ feed containing primarily 2-butene. The process comprises contacting said feed with ethylene in a metathesis reaction zone containing a metathesis catalyst under metathesis reaction conditions to provide an effluent including propylene, said metathesis catalyst consisting essentially of a transition metal or oxide thereof supported on a high purity silica support possessing less than about 150 ppm magnesium, less than about 900 ppm calcium, less than about 900 ppm sodium, less than about 200 ppm aluminum, and less than about 40 ppm iron.

### BRIEF DESCRIPTION OF THE DRAWING(S)

Various embodiments are described below with reference to the drawings wherein:

FIG. 1 is a schematic representation of the process of the invention;

FIG. 2 is a graph illustrating the metathesis reaction propylene selectivity vs. E/nB molar feed ratio for high purity 2-butene feed; and,

FIG. 3 is a graph illustrating the metathesis reaction propylene selectivity vs. E/nB molar feed ratio for low purity 2-butene feed.

### DETAILED DESCRIPTION OF PREFERRED EMBODIMENT(S)

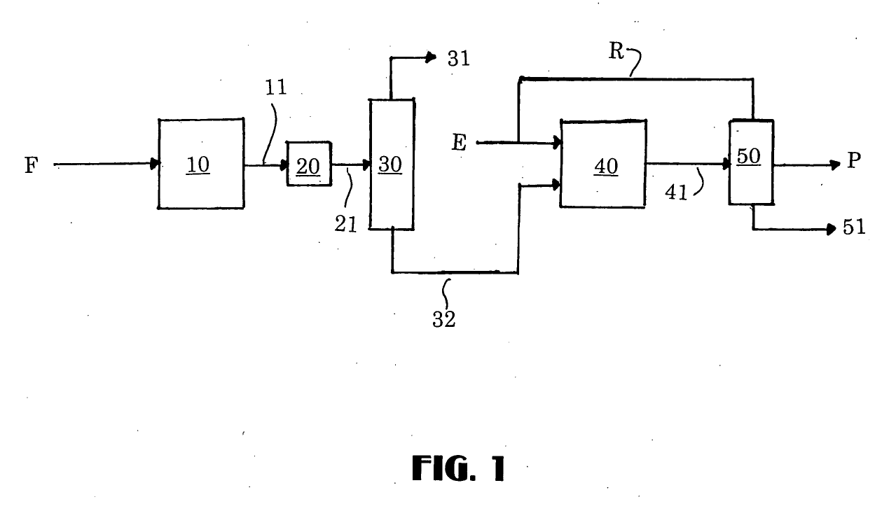

Referring to FIG. 1, the process 100 of the present invention is schematically outlined in a flow chart. The process employs the catalyst as described in U.S. Pat. No. 6,683,019 B2, which is described below in more detail.

The feed F can be a mixture of C₄ compounds such as raw steam cracker C₄'s or FCC butylenes and typically includes C₄ acetylenes, butadiene, iso and normal butenes, and iso and normal butane. A typical steam cracker C₄ cut contains components as set forth in Table 1. Table 1 is given for purposes of exemplification only. Component percentages of C₄ streams can be outside of the ranges given in Table 1.

TABLE 1

| C₄ acetylenes | trace |
|---|---|
| butadiene | 30-40 wt. percent |
| 1-butene | 10-20 wt. percent |
| 2-butene | 5-15 wt. percent |
| isobutene (isobutylene) | 20-40 wt. percent |
| iso & normal butane | 5-15 wt. percent |

The Feed F is first sent to a selective hydrogenation unit 10 for catalytically hydrogenating the C₄-acetylenes and the butadiene to 1-butene and 2-butene. Hydrogenation can be performed in a conventional manner in a fixed bed or alternately in a catalytic distillation unit. The catalytic hydrogenation unit 10 can employ any suitable hydrogenation catalyst such as, for example, palladium on alumina, in a packed bed. Hydrogen can be added at a level representing 1.0 to 1.5 times the hydrogen required to hydrogenate the dienes and acetylenes to olefins. The conditions are variable depending on reactor design. If, for example, the catalytic hydrogenation unit 10 is operated as a catalytic distillation unit, the temperature and pressure are consistent with fractionation conditions. The C₄ fraction produced by catalytic hydrogenation unit contains mainly 1-butene, 2-butene, isobutene and a small amount of other components such as normal and iso butanes. The diene content in effluent 11 from the catalytic hydrogenation unit 10 can vary depending upon downstream processing. If subsequent hydrogenation steps are anticipated, then higher amounts of dienes can leave in the selective hydrogenation effluent. In most cases, the butadiene should be reduced to less than 1500 ppm if additional hydrogenation is planned or to less than 50 ppm if no additional hydrogenation steps are planned.

Alternately, the butadiene could be removed via extraction in accordance with known procedures.

The effluent 11 of the selective hydrogenation unit 10 is optionally then sent to a fixed bed unit 20 for treatment to remove catalyst poisons such as methanol, water, mercaptans, dimethyl ether, acetaldehyde, carbonyl sulfide, acetone, t-butyl alcohol, dimethyl formamide, and peroxides. The fixed bed treater 20 preferably contains one or more beds containing a particulate adsorbent such as alumina, Y type zeolites, X type zeolites, activated carbon, alumina impregnated with Y type zeolite, alumina impregnated with X type zeolite, or a combination thereof. Alternatively, fixed bed treater unit 20 can be situated elsewhere in the process scheme provided that it is upstream of the metathesis unit 40 described below. For example, fixed bed treater unit 20 can be situated after the catalytic distillation unit 30 so as to treat the bottom butene stream 32 prior to the metathesis reactor 40.

The C₄ fraction effluent at this point will have both normal and isobutanes and butenes in addition to trace levels of C₃ and C₅ components. It is desired for the maximum production of propylene that the reaction between 2-butene and ethylene be maximized. Further, depending upon the level of butadiene removal in the selective hydrogenation step, some final butadiene removal may be necessary. If that is the case, a second hydrogenation unit is used. However, under such conditions of hydrogenation, hydroisomerization reactions also occur. Significant quantities of 2-butene are formed by the hydroisomerization of 1-butene, which both exists in the feedstock or is produced by the hydrogenation of butadiene This reaction can occur either in a separate fixed bed or in a catalytic distillation unit.

The effluent 21 of the fixed bed unit 20, contains only olefins (particularly n-butenes and isobutylene) and paraffins, and is processed for the removal of the isobutylene fraction in unit 30. There are a number of processes that will accomplish this.

In a preferred method the isobutylene is removed by catalytic distillation (“CD”) combining hydroisomerization and superfractionation in unit 30, which operates as a “de-isobutylenizer.” The hydroisomerization converts 1-butene to 2-butene, and the superfractionation removes the isobutylene which is taken off in stream 31, leaving a relatively pure 2-butene stream 32 typically containing some n-butane. An advantage to converting the 1-butene to 2-butene in this system is that the boiling point of 2-butene (1° C. for the trans isomer, 4° C. for the cis isomer) is further away from the boiling point of isobutylene (−7° C.) than that of 1-butene (−6° C.), thereby rendering the removal of isobutylene by superfractionation easier and less costly and avoiding the loss of 1-butene overhead with the isobutylene. The effluent 32 of the CD unit is sent to metathesis reactor 40.

Alternatively, the effluent from the treater 20 could be sent to a separate fixed bed unit designed to operate as a hydroisomerization unit (not shown). The effluent from that reactor, now maximized in 2-butene, can be fed to a isobutylene removal system. That system could be a superfractionator with isobutylene (and isobutane if present) going overhead. It could also be an MTBE unit or an isobutylene dimerization unit to reactively remove isobutylene. In either case, the butene effluent from the process remains essentially high in 2-butene.

The other feed to the metathesis unit is ethylene stream E. The CD unit 30 can be operated to produce highly pure 2-butene or a stream of lesser purity 2-butene. Operating the unit so as to produce lesser purity 2-butene results in savings in capital investment and operating costs at this stage of the system. However, loss of product yield in the metathesis unit 40 needs to be overcome in order to employ the lesser purity 2-butene effluent as a feed stream for the metathesis process 40 described below. Preferably, the 2-butene content of the feed to the metathesis reactor can range from about 85% to about 100%. More preferably, the 2-butene content of the feed to the metathesis reactor is at least about 90%, and most preferably at least about 95%, by weight. The metathesis unit includes the catalyst described below which enables a lower purity 2-butene stream to be used while retaining high selectivity to propylene.

Another factor to consider is the molar ratio of ethylene to the n-butenes (1- and 2-butene, cis and trans isomers), i.e., E/nB ratio, in the feed to the metathesis reactor 40. Lower E/nB ratio results in savings from lower ethylene recycle rates. Ethylene recycle is energy intensive and requires costly refrigeration. However, lower E/nB ratio can result in lower propylene selectivity. The catalyst described below provides higher propylene selectivity than those of conventional catalysts even at lower E/nB ratios. The E/nB molar ratio can range from at least about 0.5 to not more than about 4, preferably at least about 0.6 to not more than about 3, and yet more preferably from at least about 0.8 to not more than about 2.5.

More particularly now with respect to the metathesis catalyst of the invention, the high purity silica support utilized in the preparation of the metathesis catalyst of the invention possesses low amounts of both acidic and basic sites (preferably essentially no acidic and basic sites) and thereby improves the selectivity of the metathesis reaction and minimizes undesirable double bond isomerization.

By “low amount” of acidic and basic sites on the support, it is meant that the silica support possesses, by weight, less than about 150 ppm magnesium (measured as the element), less than about 900 ppm calcium (measured as the element), less than about 900 ppm sodium (measured as the element), less than about 200 ppm aluminum (measured as the element) and less than about 40 ppm iron (measured as the element). Preferably, the high purity support possesses less than about 100 ppm magnesium, less than about 500 ppm calcium, less than about 500 ppm sodium, less than about 150 ppm aluminum and less than about 30 ppm iron. More preferably, the high purity support possesses less than about 75 ppm magnesium, less than about 300 ppm calcium, less than about 300 ppm sodium, less than about 100 ppm aluminum and less than about 20 ppm iron. An example of a high purity silica within the scope of this invention that can be commercially obtained is chromatographic grade silica Other high purity silica catalyst supports can also be obtained.

Group VIA (Cr, Mo, W) and VII A (Mn, Tc, Re) transition metals and oxides thereof that can be employed herein are known and include, but are not limited to, tungsten, molybdenum, rhenium, oxides thereof and mixtures thereof. Tungsten oxide is particularly preferred. The oxides of these metals are typically formed from oxide precursors which are subsequently converted to the oxides by calcination. Suitable precursors include compounds which are convertible to the oxide form under calcination, such as, for example, the halides, oxides, sulfides, sulfates, nitrates, acetates, ammonium salts, and the like, and mixtures of any two or more thereof. Ammonium meta tungstate is preferably utilized as the precursor for the tungsten deposited upon the high purity support.

The Group VIA or VII A transition metals or oxide thereof is deposited on the high purity support material in an amount that varies between 1 and 20% by weight, based on the weight of the entire catalyst.

The high purity silica support and transition metal or oxide thereof can be contacted in any suitable manner. For example, the support and a solution containing the transition metal or oxide thereof (or precursor thereof) (hereinafter referred to simply as the transition metal) can be mixed in an open vessel, then any excess liquid can be decanted or removed by filtration. Alternatively, the technique of incipient wetness can be employed whereby only enough liquid is employed to thoroughly wet the support, with no free residual liquid. Thus, only as much transition metal-containing solution is employed as the support can absorb. This can be accomplished, for example, by spraying the solution over a quantity of support which is being tumbled in a rotating, baffled drum. Such treatment can also be carried out by simply pouring a predetermined quantity of the solution over a quantity of the silica support in an open vessel. Alternatively, a measured quantity of support could be added to a volume of transition metal-containing solution such that all of the liquid is imbibed by the added support. Other techniques are known to those skilled in the art and can also be employed. For example, a quantity of support may be placed in a tubular reactor, a volume of transition metal-containing solution may be percolated there through, followed by further treatment/activation as necessary.

The conditions of high purity silica support/transition metal-containing solution contacting are not critical. Any temperature and any period of contact time are suitable. For convenience, contacting is generally carried out at about room temperature, although higher or lower temperatures can be employed. A time period sufficient to allow the support and reagents to come into intimate contact is all that is necessary. Thus, the support and solution may be brought into contact for as little time as a few seconds to several hours or more, as convenient.

Following contact of the high purity silica support and transition metal-containing solution, any excess liquid can be removed by suitable means, such as, for example, decantation, filtration or the like. The treated support can be dried to removed absorbed solvent. Any suitable means, as well known by those skilled in the art, may be employed such as, for example, oven drying, passing a vigorous stream of dry (moisture-free) gas over the treated support and the like. For example, the supported catalyst can be dried by heating at an elevated temperature of, e.g., about 200° C. or higher by passage of an inert gas such as nitrogen over the material. This can be accomplished within the reactor or in other suitable catalyst preparation equipment.

Calcination, when used, is conducted by heating the transition metal oxide or precursor thereof in the presence of an oxygen-containing gas, such as, for example, air, under conditions sufficient to activate the metal oxide, e.g., tungsten oxide, or to convert the transition metal compound present, e.g., tungsten, to the activated metal oxide form. Temperatures in the range of about 350° C. to about 800° C. are generally satisfactory for such calcinations. The time for subjecting the transition metal oxide to calcination is an amount of time sufficient to activate the catalyst. Anywhere from a few minutes to several hours is suitable. Typically, about 15 minutes to about 20 hours of calcination will be sufficient. Preferably, for the most efficient use of reaction equipment, the transition metal oxide will be subjected to calcination for about 30 minutes to about 6 hours at temperatures less than 650° C. Higher temperatures while acceptable can result in loss of support surface area and reduction in catalyst activity. Typically less time is required at higher temperatures and vice versa.

After calcination, the metathesis catalyst is optionally treated under reducing conditions such as, for example, with carbon monoxide, hydrogen, or a hydrocarbon at a temperature in the range of from about 350° C. to about 550° C. to enhance the metathesis activity of the catalyst. Such reducing treatment is carried out preferably in the range of from about 400° C. to about 450° C., because good catalyst activation with relatively short activation periods of about one to about six hours can be achieved. Such optional reducing treatment can suitably be carried out for a period of time ranging from about 1 minute to about 30 hours. If desired, the calcined catalyst can be further treated with an inert gas such as nitrogen prior to use in a metathesis reaction to remove adsorbed materials from the catalyst which may have a detrimental effect on the selectivity of the catalyst for metathesis reactions. Such materials are water or CO₂ that could be adsorbed by the catalyst through contact with the ambient environment.

The metathesis catalyst produced has a minimum of active sites that promote isomerization. Importantly, unlike the production of propylene using the conventional catalysts, when utilizing feeds with a high concentration of 2-butene, the metathesis catalyst utilized herein should not be intentionally admixed with double bond isomerization catalysts, including supported or unsupported phosphoric acid, bauxite, zinc oxide, magnesium oxide, calcium oxide, cerium oxide, thorium oxide, titanium oxide, cobalt oxide, iron oxide, or manganese oxide, and the like, since such isomerization catalysts will significantly interfere with the desired metathesis reaction.

The metathesis reaction conditions in accordance with the invention include a temperature of from about 50° C. to about 600° C., preferably from about 200° C. to about 400° C., a weight hourly space velocity (WHSV) of from about 3 to about 200, preferably from about 6 to about 40, and a pressure of from about 10 psig to about 600 psig, preferably from about 30 psig to about 100 psig. The reaction may be carried out by contacting the olefin(s) with the catalyst in the liquid phase or the gas phase depending on structure and molecular weight of the olefin(s). If the reaction is carried out in the liquid phase, solvents or diluents for the reaction can be used. Aliphatic saturated hydrocarbons, e.g., pentanes, hexanes, cyclohexanes, dodecanes and aromatic hydrocarbons such as benzene and toluene are suitable. If the reaction is carried out in the gaseous phase, diluents such as saturated aliphatic hydrocarbons, for example, methane, ethane, and/or substantially inert gases, e.g., nitrogen, argon, can be present. Preferably, for high product yield, the reaction is conducted in the absence of significant amounts of deactivating materials such as water and oxygen.

The contact time needed to obtain a desirable yield of metathesis reaction products depends upon several factors such as the activity of the catalyst, temperature, pressure, and the structure of the olefin(s) to be metathesized. Length of time during which the olefin(s) are contacted with catalyst can conveniently vary between 0.1 seconds and 4 hours, preferably from about 0.5 sec to about 0.5 hrs.

The process can be conducted batch-wise or continuously with fixed catalyst beds, slurried catalyst, fluidized beds, or by using any other conventional contacting techniques.

The effluent 41 from the metathesis reactor 40 is sent to a separation operation 50 including one or more separation units such as distillation columns and the like. Propylene product P is removed. Ethylene is recovered and recycled via recycle stream R back to the ethylene feed stream E to the metathesis reactor 40. Refrigeration requirements make the ethylene recycle energy intensive. Hence, minimizing ethylene requirements with its concomitant costs can provide economic savings. Other products, such as butane and unconverted C₄'s, and other components can be removed via line 51. The propylene product P is typically used as a monomer for the manufacture of polypropylene homopolymers and copolymers.

Various features of the invention are illustrated by the examples presented below.

A series of tests was made to evaluate the effects of varying E/nB molar feed ratios on propylene selectivity using the preferred metathesis catalyst having a high purity silica support as per U.S. Pat. No. 6,683,019 with comparative data for a conventional state-of-the-art metathesis catalyst having a low purity silica support. The conventional catalyst support typically contains about 60-325 ppm magnesium (measured as the element); 360-1660 ppm calcium (measured as the element); 760-1450 ppm sodium (measured as the element); 245-285 ppm aluminum (measured as the element); and 30-85 ppm iron (measured as the element). Said materials are available from several commercial silica producers. Raw steam cracker C₄'s or FCC butylenes are first processed through a first stage selective hydrogenation unit to reduce the diene content to less than about 50 ppmw. The effluent is then passed through treaters for poison removal and the recovered C₄'s after treatment are then subjected to either an integrated fixed bed hydroisomerization/de-isobutylenizer or a catalytic distillation-based de-isobutyleneizer (referred to as “CD-DIB”) wherein the contained 1-butene is hydroisomerized to 2-butene and the fractionation takes a high purity isobutylene stream as overhead product and a high purity 2-butene stream as a bottoms product. The fractionator can be designed to control the exact composition of the bottoms product which will be dictated by the economic tradeoff between capital investment/operating costs and product yields.

A series of tests was made with feedstocks processed at two different DIB or CD-DIB fractionator design conditions. Series I (Example 1 and Comparative Example A) used a 2-butene stream having a composition corresponding to essentially high purity 2-butene (ca., 99+%), which is typical for CD-DIB designs that simultaneously maximize isobutylene recovery and 2-butene recovery. The high purity of the CD-DIB bottoms stream, when integrated with a metathesis unit using the preferred catalyst with the high purity silica support, allows the use of a smaller metathesis reactor and less metathesis catalyst inventory by virtue of not needing to admix an isomerization catalyst, e.g., MgO, which would otherwise be required to maximize propylene yield if there was an appreciable quantity of 1-butene in the 2-butene-rich CD-DIB bottoms stream.

Series II tests (Example 2 and Comparative Example B) used a 2-butene stream having a composition equivalent to 91% 2-butene, 5% 1-butene, and 4% isobutylene. This design results in lower capital costs and energy costs for the CD-DIB but at the expense of loss of the valuable 2-butene product which in the downstream metathesis unit reacts with ethylene to result in the highest selectivity to propylene.

### EXAMPLE 1

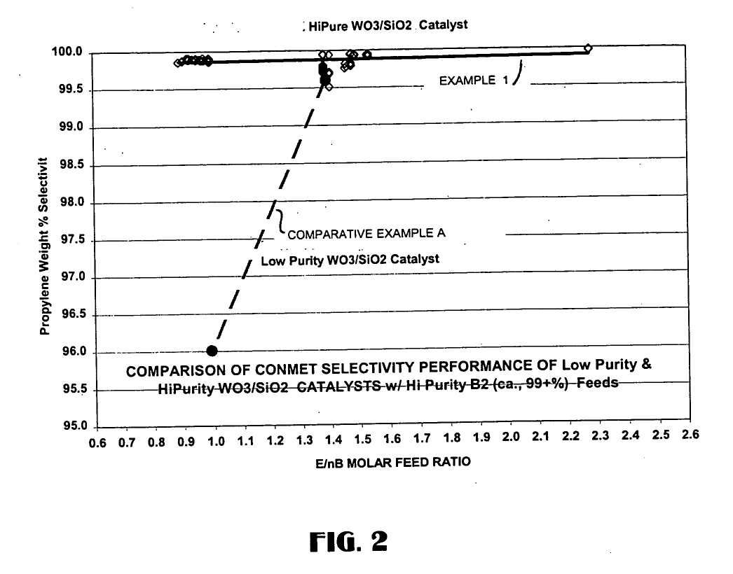

A feed containing high purity (99+% by weight) 2-butene along with ethylene was introduced into a metathesis reactor containing a catalyst containing 7.7% by weight WO₃ on a high purity silica support, synthesized in accordance with the method of the invention described herein above. The metathesis reaction was conducted at a temperature of 350° C., a pressure of 350 psig, and a WHSV of 14 employing a range of E/nB molar ratios. The propylene weight % selectivity was calculated and plotted in FIG. 2. The propylene selectivity remained at close to 100% for the entire range of E/nB ratios (i.e., from E/nB 0.8-2.3).

### COMPARISON EXAMPLE A

A metathesis reaction was conducted with a high purity 2-butene feed in accordance with the method of Example 1 except that a commercial low purity WO₃/SiO₂ catalyst was employed. The propylene selectivity results are plotted in FIG. 2 and show a dramatic drop of propylene selectivity going down from 99.5 to 96.0 as the E/nB ratio drops from 1.4 to 1.0.

### EXAMPLE 2

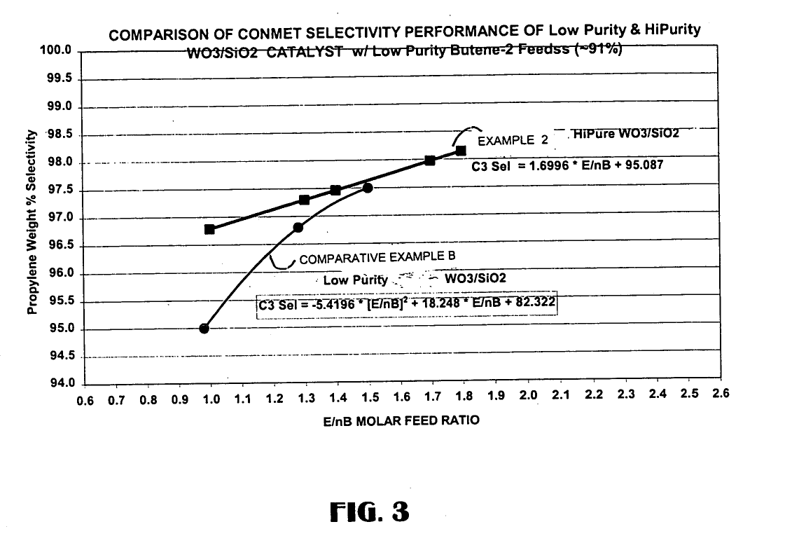

A metathesis reaction was conducted using the catalyst of the invention in accordance with the method of Example 1 except that a low purity feed was employed containing 91% 2-butene, 5% 1-butene, and 4% isobutylene, by weight. The propylene selectivity over a range of E/nB molar ratios was determined and plotted. The results are shown in FIG. 3. The propylene selectivity ranged from a high of 98.2 at an E/nB ratio of 1.8 to a low of 96.75 at an E/nB ratio of 1.0.

### COMPARATIVE EXAMPLE B

A metathesis reaction was conducted in accordance with the method of Example 2 except that the low purity commercial catalyst was used. The propylene selectivity dropped from 97.5 to 95.0 as the E/nB ratio dropped from 1.5 to 1.0.

These results show the unexpected superiority of the catalyst of the invention for the production of propylene by the metathesis reaction between ethylene and 2-butene as opposed to commercial low purity catalysts over a range of E/nB ratios, even when using low purity 2-butene feeds and at low E/nB ratios. Considerable cost savings can be achieved by reduced ethylene recycle costs and reduced capital and operating expenses for the catalytic distillation unit for removing isobutylene from the feed.

With respect to Example 1 and Comparative Example A, over the range of 0.8 to 2.3 E/nB molar feed ratio, the metathesis catalyst with the preferred high purity silica support of the invention (Example 1) exhibited very high propylene selectivity, ca., 99+% and the selectivity does not change as E/nB ratio is decreased. The prior art metathesis catalyst with the conventional low purity silica support (Comparative Example A) showed high propylene selectivities at E/nB feed ratios in excess of about 1.5 but selectivity drops off markedly as E/nB is decreased from 1.5 to the 1.0 level. Thus, the metathesis catalyst with the high purity silica support exhibits almost a 4 point propylene selectivity advantage over that of the state-of-the-art metathesis catalyst while allowing operation at very low ethylene recirculation rates, ca., 1.0 E/nB molar. With respect to Example 2 and Comparative Example B, over the range of 1.0 to 1.8 E/nB molar feed ratio, the metathesis catalyst with the preferred high purity silica support (Example 2) exhibited a linear relationship between propylene selectivity and E/nB ratio with a slope equivalent to about 1.7 propylene selectivity units per unit change in E/nB molar feed ratio. The metathesis catalyst with the conventional state-of-the-art low purity silica support (Comparative Example B) exhibited a parabolic decline in propylene selectivity as E/nB molar feed ratio was decreased. Over the narrow range of E/nB values of 1.0 to 1.2, the decline in propylene selectivity for the metathesis catalyst with the state-of-the-art low purity silica support is near linear and approximately 5 times greater than that for the preferred metathesis catalyst with the high purity silica support as seen in FIG. 3.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

1. A process for producing propylene from a C₄ feed containing 2-butene comprising contacting said feed with ethylene in a metathesis reaction zone containing a metathesis catalyst containing oxides of group VIA or VIIA metals under metathesis reaction conditions to provide an effluent including propylene, said metathesis catalyst consisting essentially of a transition metal or oxide thereof supported on a high purity silica support possessing less than about 150 ppm magnesium, less than about 900 ppm calcium, less than about 900 ppm sodium, less than about 200 ppm aluminum, and less than about 40 ppm iron.

2. The process of claim 1 wherein the high purity silica support possesses less than about 100 ppm magnesium, less than about 500 ppm calcium, less than about 500 ppm sodium, less than about 150 ppm aluminum, and less than about 30 ppm iron.

3. The process of claim 1 wherein the high purity silica support possesses less than about 75 ppm magnesium, less than about 300 ppm calcium, less than about 300 ppm sodium, less than about 100 ppm aluminum, and less than about 20 ppm iron.

4. The process of claim 1 wherein the molar ratio of ethylene to n-butenes in the feed is at least about 0.5 and not more than about 4.

5. The process of claim 1 wherein the molar ratio of ethylene to n-butenes in the feed is at least about 0.6 and not more than about 3.

6. The process of claim 1 wherein the molar ratio of ethylene to n-butenes in the feed is at least about 0.8 and not more than about 2.5.

7. The process of claim 1 wherein the feed contains at least about 85% by weight of 2-butene.

8. The process of claim 1 wherein the feed contains at least about 90% by weight of 2-butene.

9. The process of claim 1 wherein the feed contains at least about 99% by weight of 2-butene.

10. The process of claim 1 wherein the transition metal oxide of the catalyst is tungsten oxide.

11. The process of claim 1 wherein the effluent from the metathesis reaction zone includes unreacted ethylene and the process further comprises recovering propylene product from the effluent and separating and recycling at least some of the unreacted ethylene to the metathesis reaction zone.

12. The process of claim 1 wherein the feed is derived from one or more source selected from the group consisting of steam cracker butylenes, FCC butylenes, MTBE raffinate, polybutylene raffinate and polyisobutylene raffinate.

13. The process of claim 8 wherein the weight percent selectivity to propylene exceeds 96%.

14. The process of claim 9 wherein the weight percent selectivity to propylene exceeds 99.5%.

15. A process for producing propylene from a hydrocarbon fraction containing unsaturated C₄ compounds, comprising:
a) subjecting the hydrocarbon fraction to selective hydrogenation to convert at least some C₄ acetylene and butadiene components and provide an effluent containing isobutylene, 1-butene and 2-butene;
b) subjecting the effluent from the selective hydrogenation step simultaneously or in series to (i) an isobutylene removal step to remove at least a major portion of the isobutylene and (ii) a hydroisomerization step to convert at least a major portion of the 1-butene to 2-butene so as to provide an effluent, a major portion of the effluent being 2-butene;
c) contacting the effluent from the catalytic distillation hydroisomerization step (ii) with ethylene in a metathesis reaction zone containing a metathesis catalyst under metathesis reaction conditions to provide an effluent including propylene, said metathesis catalyst consisting essentially of a Group VIA or VIIA transition metal or oxide thereof supported on a high purity silica support possessing less than about 150 ppm magnesium, less than about 900 ppm calcium, less than about 900 ppm sodium, less than about 200 ppm aluminum, and less than about 40 ppm iron.

16. The process of claim 15 further including the step of removing catalyst poison from any one or more of the hydrocarbon fraction, the selective hydrogenation effluent or the catalytic distillation effluent.

17. The process of claim 16 wherein the step of removing catalyst poison is conducted in a fixed bed containing a particulate sorbent material.

18. The process of claim 16 wherein the sorbent material is selected from the group consisting of alumina, Y type zeolites, X type zeolites, activated carbon, alumina impregnated with Y type zeolite, alumina impregnated with X type zeolite and combinations thereof.

19. The process of claim 15 wherein the high purity silica support possesses less than about 100 ppm magnesium, less than about 500 ppm calcium, less than about 500 ppm sodium, less than about 150 ppm aluminum, and less than about 30 ppm iron.

20. The process of claim 15 wherein the high purity silica support possesses less than about 75 ppm magnesium, less than about 300 ppm calcium, less than about 300 ppm sodium, less than about 100 ppm aluminum, and less than about 20 ppm iron.

21. The process of claim 15 wherein the molar ratio of ethylene to n-butenes in the feed is at least about 0.5 and not more than about 4.

22. The process of claim 15 wherein the molar ratio of ethylene to n-butenes in the feed is at least about 0.6 and not more than about 3.

23. The process of claim 15 wherein the molar ratio of ethylene to n-butenes in the feed is at least about 0.8 and not more than about 2.5.

24. The process of claim 15 wherein the feed contains at least about 85% by weight of 2-butene.

25. The process of claim 15 wherein the feed contains at least about 90% by weight of 2-butene.

26. The process of claim 15 wherein the feed contains at least about 99% by weight of 2-butene.

27. The process of claim 15 wherein the transition metal oxide of the catalyst is tungsten oxide.

28. The process of claim 15 wherein the effluent from the metathesis reaction zone includes unreacted ethylene and the process further comprises recovering propylene product from the effluent and separating and recycling at least some of the unreacted ethylene to the metathesis reaction zone.

29. The process of claim 15 wherein the metathesis reaction conditions include a temperature of from about 50° C. to about 600° C., a WHSV of from about 3 to about 200 and a pressure of from about 10 psig to about 600 psig.

30. The process of claim 25 wherein the weight percent selectivity to propylene exceeds 96%.

31. The process of claim 26 wherein the weight percent selectivity to propylene exceeds 99.5%.

32. The process of claim 31 wherein the molar ethylene to n-butenes feed ratio at the inlet of the metathesis reactor is at least about 0.9 and not more than about 2.5.
